(19) United States
(12) Patent Application Publication
(10) Pub. No.: US 2014/0088405 A1
(43) Pub. Date: Mar. 27, 2014

(54) METHOD AND MEDICAL IMAGING DEVICE TO DETERMINE AND DISPLAY AN EMOTIONAL STATE OF A PATIENT DURING THE MEDICAL IMAGING

(71) Applicants: Stefan Assmann, Erlangen (DE); Susanne Bay, Erlangen (DE)

(72) Inventors: Stefan Assmann, Erlangen (DE); Susanne Bay, Erlangen (DE)

(21) Appl. No.: 14/039,378

(22) Filed: Sep. 27, 2013

(30) Foreign Application Priority Data
Sep. 27, 2012 (DE) 102012217645.4

(51) Int. Cl. A61B 5/16; A61B 5/053; A61B 5/055

(52) U.S. Cl. CPC A61B 5/165; A61B 5/055; A61B 5/053
USPC 600/411

(57) ABSTRACT

In a medical imaging method and apparatus, an emotional state of a patient is recorded during a medical imaging examination, by recording at least one emotion parameter of the patient during the medical imaging examination, and calculating an emotional state of the patient using the at least one emotion parameter of the patient during the medical imaging examination.

### BACKGROUND OF THE INVENTION

1. Field of the Invention

The present invention relates to a method and medical imaging device for recording an emotional state of a patient during a medical imaging examination.

2. Description of the Prior Art

For medical imaging examinations, such as a magnetic resonance examination or a computed tomography examination and/or a radiological examination, for achieving high imaging quality it is necessary that the patient lie as still, i.e., as motionless, as possible, and assume a still and/or relaxed position. The patient should assume this still and/or relaxed position for the entire duration of the medical imaging examination, which may last up to an hour.

Medical imaging devices, in particular magnetic resonance devices and/or computed tomography devices and/or radiological imaging devices, frequently have a cylindrical patient reception area in which the patient is positioned for the medical imaging examination. However, due to the narrowness in this cylindrical patient reception area the patient can experience uneasiness and/or anxiety states. The uneasiness and/or the anxiety states of the patient can in addition elicit a physiological reaction from the patient, such as for example accelerated breathing and/or increased and/or a desire to void one's bladder and/or increased muscle tone and/or dry mouth, etc. In addition, this physiological reaction of the patient can lead to restlessness of the patient and/or to tremors of the patient and/or additional, in particular uncontrolled, movements of the patient, which can have a negative impact on the medical imaging examination. Frequently this state of the patient is unobserved until it is too late, so that medical image data are acquired during which the patient has made an undesired movement. This can lead to a repetition of at least portions of the medical imaging examination, so that the examination time for the medical imaging examination prolonged and the patient has a longer length of stay in the patient reception area. This longer length of stay in the patient reception area and/or the repetition of at least partial measurements of the medical imaging examination can also have as its consequence a higher exposure to radiation for the patient.

Conventionally, medical imaging devices have an emergency call device such as an “emergency ball”, by means of which the patient can communicate an emergency signal to the hospital technician during the medical imaging examination.

### SUMMARY OF THE INVENTION

The present invention addresses the problem of providing a method and/or a device in which a monitoring of the emotional state of the patient takes place during a medical examination.

In a method for recording an emotional state of a patient during a medical imaging examination in accordance with the invention, at least one emotion parameter of the patient is recorded during the medical imaging examination and a calculation of an emotional state of the patient is made during the medical imaging examination using the at least one emotion parameter of the patient.

As a result of this method, it is possible to record an emotional state of the patient in particularly advantageous manner, with the recording being independent of a self-evaluation and/or a self-assessment by the patient with respect to his or her emotional state. Preferably the emotion parameters are continuously recorded during the entire medical imaging examination, so that during the entire examination period a current value of an emotional state is always available for the medical imaging examination. In addition, an advantageous supervision and/or monitoring of the patient can take place during the medical imaging examination and/or it is possible to respond to changes in the emotional state of the patient. For example, in the case of critical values of the emotional state of the patient a measurement strategy can be altered and/or also at least a partial measurement of the medical imaging examination can be cancelled. The calculated emotional state of the patient can for example be a stress value of the patient that is determined from the recorded emotion parameters. In this connection, an emotion parameter should be understood as a parameter that has a value that is dependent on an emotion felt by the patient.

In an embodiment, the emotion parameter is a conductivity parameter recorded on the skin surface of the patient. Preferably in this connection an electrical conductivity of the patient is recorded, with the electrical conductivity of the skin corresponding to a skin resistance. This embodiment of the inventive method makes it possible in advantageous manner to record an emotion parameter of the patient especially reliably. The value of the electrical conductivity is, for example, influenced by activity of the sweat glands of the patient. Activity of the sweat glands is controlled by the autonomic nervous system of the patient, hence cannot be consciously influenced by the patient. In addition, the recording of a conductivity parameter constitutes a particularly low impact to the patient. As an alternative or in addition, the recorded emotion parameter can be a blood pressure parameter and/or a pulse parameter. Additional emotion parameters appearing to be useful to a person skilled in the art can be recorded.

In another embodiment, the calculated emotional state of the patient is represented graphically. Preferably the calculated emotional state, in particular a calculated stress value, is represented on a monitor for the medical imaging examination hospital technician, for example a physician supervising the medical imaging examination. As a result, a hospital technician supervising the medical imaging examination can always obtain exact information about a current emotional state of the patient and if necessary take counter-measures and/or protective measures. In addition, in this way a risk of recording useless and/or blurred data and/or a risk of the existence of a critical situation for the patient, can be particularly well assessed and/or recorded by the medical service personnel. In addition, the representation of the calculated emotional state can take place in a control room, which is constructed separately from an examination room, as is of advantage in the case of magnetic resonance examinations and/or radiological examinations.

In a further embodiment of the invention a sedation step is executed depending on a value of the calculated emotional state of the patient. The emotional state of the patient thus can be actively influenced. Actions can be then taken that have a calming effect on the patient. These measures, in particular the sedation step, can be executed before the patient performs an uncontrolled movement due to his or her emotional state.

Furthermore, in the sedation step an adjustment and/or a change of measurement parameters can take place depending on the value of the calculated emotional state of the patient. In this way additional stress situations of the patient during the further medical imaging examination can be prevented and a stable, calm state of the patient can be brought about, so that there are optimal conditions for the recording of medical image data. In addition, individual measurement parameters can be adapted to the emotional state of the patient, for example a sequence within a scan protocol can be adapted to the emotional state of the patient and/or a whisper mode can be activated, preferably for a magnetic resonance examination. For example, in this connection a scan protocol adapted to the emotional state of the patient can be more de-sensitized with respect to a movement of the patient and/or make possible a more rapid measurement sequence, so that the stressful situation for the patient can be shortened. In addition, the adjustment and/or change of measurement parameters can lead to a pause in the measurement. Further, an adjustment and/or change of the Dot (day optimizing throughput) engine strategy are possible. A Dot engine strategy is a computerized scheduling routine that optimizes the daily workflow for making use of (occupying) imaging installations.

Especially advantageously in the sedation step, a portion of the data acquisition by the medical imaging examination can be cancelled. This can be advantageous, in particular in the event of a panic attack and/or in the event of an impending panic attack of the patient and/or extreme stress situations of the patient, since here the medical imaging examination and/or a portion of the medical imaging examination can be cancelled prematurely. In addition, in especially critical situations the patient can be removed from the patient reception area for the purpose of calming. For such a measure the emotional state, in particular the calculated stress value of the patient, is especially high, such as greater than a critical threshold value. Due to such an acute emotional state identified by the calculated stress value of the patient, powerful movements during a continuation of the medical imaging examination are to be expected and/or useless medical image data is likely to be recorded. Particularly in the case of computed tomography examinations and/or radiological imaging examinations, an increased exposure to radiation for the patient can be prevented as a result of this cancellation.

In addition, in the sedation step, an adjustment and/or a change of the environment of the patient can take place depending on the value of the calculated emotional state of the patient. This can have an especially calming effect on the patient during the medical imaging examination, so that the emotional state, in particular the stress value of the patient, can be kept in a low range and/or the patient can assume a calm and relaxed position. For example, in this connection calming music can be played in the patient reception area and/or the patient may listen to the calming music using headphones. In addition, the lighting of the patient reception area can be changed. For example, in this connection a lighting color is selected based on the emotional state of the patient. Experimental data has shown that particular lighting colors can have a positive effect on the emotional state of the patient. In addition, the medical imaging examination hospital technician can speak in a calming manner to the patient and/or a change of the ventilation of the patient reception area can have a calming effect on the emotional state of the patient.

In a further embodiment of the invention, the sedation step can be started manually by a technician, as a result of which a particularly individualized adjustment and/or change of the patient's environment and/or of measurement parameters can be achieved. In addition, in this way a subjective impression that the patient has made on the hospital personnel can be taken into consideration in the initiation of the sedation step.

In another embodiment, an instruction (prompt) for the execution of the sedation step is represented graphically, depending on the value of the calculated emotional state of the patient. In this way the hospital technician supervising the medical imaging examination can have possible sedation steps and/or useful sedation steps pointed out visually in an especially simple manner. Further, an incorrect selection of a sedation step due to a misunderstanding of the current situation can be prevented by the hospital technician. For example, in the event of a stressful situation of the patient, for which the value of the emotional state still indicates a non-critical stress level, an instruction for the adjustment and/or a change of the patient's environment to the hospital technician can take place and/or be displayed. If the value of the emotional state indicates a high stress level, for example an instruction for the adjustment and/or a change of measurement parameters and/or for cancellation of at least a partial measurement of the medical imaging examination can be displayed. Preferably the instruction can be determined automatically depending on the value of the emotional state by a control device of the medical imaging device as well as being automatically graphically represented by the control device via a monitor for the hospital technician.

Especially advantageously, the sedation step executed depending on the value of the calculated emotional state of the patient can be executed at least partially automatically by means of the control device. In this connection a particularly rapid and direct control of sedation steps can take place. This is particularly advantageous in the case of medical imaging examinations within a clinic when the clinic personnel supervising the medical imaging examination have a high workload.

In addition, the invention includes a medical imaging device with a detector unit, a patient reception area, a control device and a sensor unit that is designed to record an emotion parameter of the patient during a medical imaging examination, and wherein the control device and/or the sensor unit is/are designed for the execution of the method for recording an emotional state of a patient according to the invention, as described above. As a result it is possible to record an emotional state of the patient, with the recording in this connection being independent of a self-evaluation or a self-assessment of the patient with respect to his or her emotional state. Preferably the emotion parameters are continuously recorded by the sensor unit during the entire medical imaging examination, so that during the entire examination period a current value of an emotional state is always available for the medical imaging examination. Preferably, the emotion parameters are recorded by the sensor unit and evaluated by the control device in order to calculates the emotional state, in particular a stress value of the patient by means of the emotion parameters. Alternatively, the sensor unit can have its own evaluation unit designed for evaluation of the recorded emotion parameters, separate from the control device.

The medical imaging device may have an emergency call device arranged within the patient reception area during the medical imaging examination, and the emergency call device may include the sensor unit for recording the emotion parameter. As a result, the sensor unit can be arranged within the medical imaging device in a space saving manner, in particular within the patient reception area. Preferably the patient holds the emergency call device in his or her hand during the medical imaging.

The sensor unit can be a conductivity sensor unit, which produces a particularly reliable recording of an emotion parameter, which is independent from a self-assessment of the patient with respect to his or her emotional state.

### BRIEF DESCRIPTION OF THE DRAWINGS

FIG. 1 is a flowchart of an inventive method for recording an emotional state of a patient in accordance with the invention.

FIG. 2 illustrates a medical imaging device in accordance with the invention.

### DESCRIPTION OF THE PREFERRED EMBODIMENTS

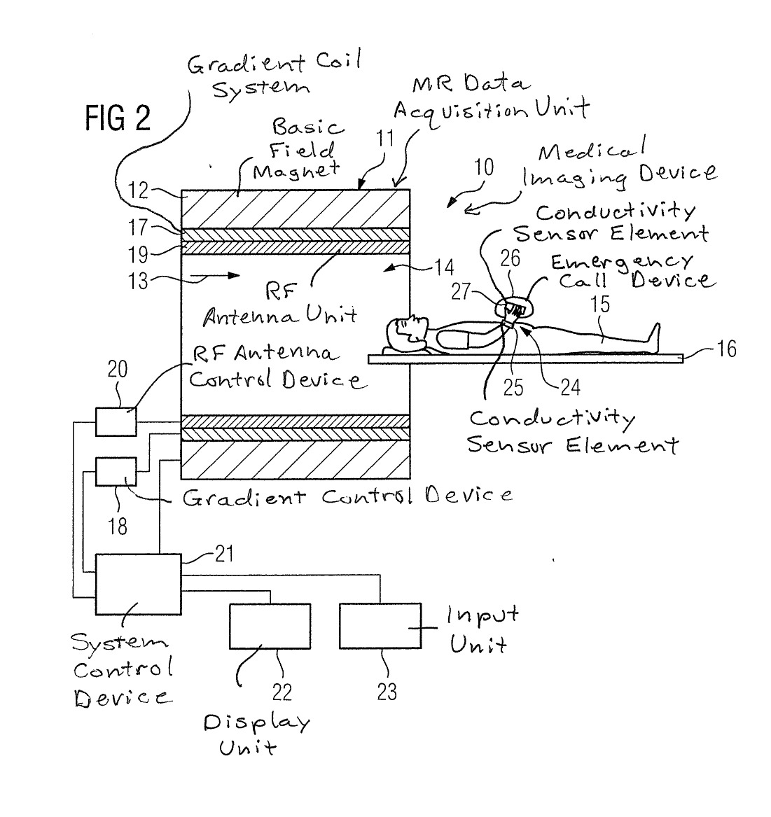

FIG. 2 shows an inventive medical imaging device 10, which in the present exemplary embodiment is formed by a magnetic resonance device. However, the invention is not limited to a design of the medical imaging device 10 as a magnetic resonance device. The medical imaging device 10 can also be formed by a computed tomography device and/or a PET device (Positron Emission Tomography device) and/or radiological imaging device, etc.

The magnetic resonance (MR) device has an MR data acquisition unit 11, which is formed by a magnet unit. The MR data acquisition unit has a basic field magnet 12 for generating a strong and constant basic magnetic field 13. In addition, the MR data acquisition unit 11 has a cylindrical patient reception area 14 for accommodation of a patient 15. The patient reception area 14 is enclosed by the MR data acquisition unit 11 in a circumferential direction. The patient 15 can be moved into the patient reception area 14 by means of an a patient bed 16 of the magnetic resonance device.

The MR data acquisition unit 11 further has a gradient coil unit 17 for generating magnetic field gradients, said magnetic field gradients being used for a local coding during imaging. The gradient coil unit 17 is controlled by a gradient control device 18 of the magnetic resonance device. In addition, the magnet unit has a radio-frequency (RF) antenna unit 19 firmly integrated within the magnet unit and a RF antenna control device 20 for exciting a polarization, which ensues in the basic magnetic field 13 generated by the basic field magnet 12. The RF antenna unit 19 is controlled by the RF antenna control device 20 and radiates RF magnetic resonance sequences in an examination volume which is essentially formed by the patient reception area 14. As a result of this the magnetization is deflected from its equilibrium position.

The magnetic resonance device has a system control device 21 formed by a processing unit for controlling the basic field magnet 12, the gradient control device 18 and for controlling the high frequency antenna control device 20. The system control device 21 centrally controls the magnetic resonance device, for example the carrying out of a predetermined imaging magnetic field gradient echo sequence. Control information, such as for example imaging parameters, as well as reconstructed magnetic resonance images can be displayed on a display unit 22, for example a monitor, of the magnetic resonance device. In addition, the magnetic resonance device has an input unit 23, by means of which information and/or parameters can be input by an operator during a measurement process.

In conventional magnetic resonance devices, the patient reception area 14 ordinarily has an opening cross-section of about 50 cm to a maximum of 70 cm. A patient 15 arranged and/or positioned within the patient reception area 14 can suffer anxiety states and/or stressful situations due to the restricted cross-section of the patient reception area 14. The medical imaging device 10 has a sensor unit 24 for recording an anxiety state and/or a stressful situation of the patient 15, said sensor unit being designed to record an emotion parameter of the patient 15 during the medical imaging examination. To this end the sensor unit 24 is formed by a conductivity measurement device that records an electrical conductivity of the patient 15, in particular of a skin surface of the patient 15. At least one conductivity sensor element 25, formed by a conductivity probe, is arranged at a hand region and/or arm region of the patient 15, so that during the entire medical imaging examination there is always contact between the conductivity sensor element 25 and the skin of the patient 15.

In addition, the medical imaging device 10 has an emergency call device 26 which in the present exemplary embodiment is formed by an emergency ball. The emergency call device 26 is a communication means, by means of which the patient 15 can transmit an emergency signal to a hospital technician supervising the medical imaging examination, in particular a physician. To this end, the patient 15 is encouraged to hold the emergency call device in his or her hand during the entire medical imaging examination, in order to prevent undesired movements, in particular hand movements, during the medical imaging examination.

An additional conductivity sensor element 27 of the sensor unit 24 is arranged in this emergency call device 26, wherein the conductivity sensor element 27 is integrated within a surface of the emergency call device, so that during the medical imaging examination 10, in particular the magnetic resonance examination, there is always contact between the conductivity sensor element 27 and the skin of the patient 15.

In an alternative embodiment of the sensor unit 24 it can have only conductivity sensor elements 25, 27 or also more than two conductivity sensor elements 25, 27. In addition, provision can be made that the sensor unit 24 has additional sensor elements that are designed for recording an additional emotion parameter, such as a sensor for recording blood pressure signals and/or for recording pulse signals, etc.

The signals or emotion parameters recorded by the sensor unit 24 are transferred after recording by a data transfer unit not presented in detail to the system control device 21 and evaluated there. To this end the data transfer unit is designed for cordless and/or wireless data transfer between the sensor unit 24 and the system control device 21. As an alternative the data transfer can also take place by means of a data transfer cable.

In addition, the medical imaging device 10 has a lighting device (not shown in detail) and a communication unit (also not shown in detail). The lighting device is designed for lighting and/or illumination of the patient reception area 14. The communication unit is designed for communication, in particular for transmitting speech signals from the medical service personnel to the patient 15, and for playing background music.

The sensor unit 24 and the system control device 21 are presently designed for the execution of an inventive method for recording an emotional state of a patient 15 during the medical imaging examination, in particular the magnetic resonance examination.

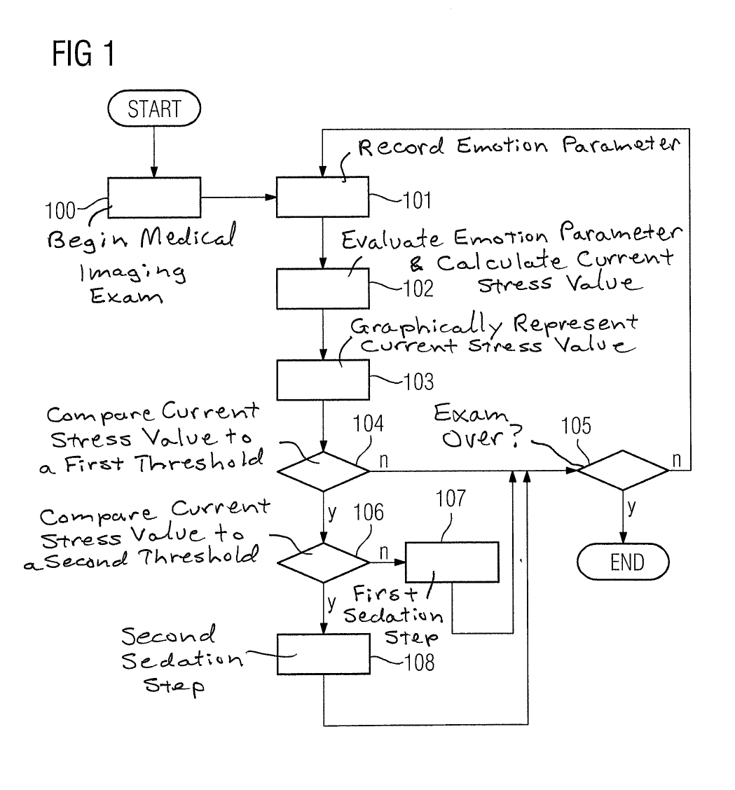

The inventive method for recording the emotional state of the patient 15 during the medical imaging examination is schematically represented in FIG. 1. Prior to beginning the method the patient 15 is prepared for the upcoming medical imaging examination, in the present exemplary embodiment for an upcoming magnetic resonance examination by a hospital technician supervising the magnetic resonance examination, in particular a physician or a medical assistant. To this end the patient 15 is positioned on the apparatus for supporting a patient 16 and accessory units that are necessary for the magnetic resonance examination, for example such as local coils and/or an EKG unit and the sensor unit 24, are positioned on the patient 15.

Then the patient 15 is brought, together with the patient bed 16, into the patient reception area 14. After that, in a first step 100 of the inventive method the medical imaging examination starts. Simultaneously with the start of the medical imaging examination, in a further step 101 the recording of an emotion parameter starts, said emotion parameter presently being formed by a conductivity value of the skin of the patient 15.

The emotion parameters recorded by the sensor unit 24, in particular the conductivity sensor elements 25, 27, are then transmitted by the data transfer unit to the system control device 21 and evaluated in the system control device 21 in a further step 102. To this end the system control device 21 has an evaluation unit not presented in detail. The system control device 21 also has the necessary software and/or computer programs for the evaluation of the recorded emotion parameters.

The system control device 21 calculates the emotional state from the emotion parameters of the patient 15 in step 102, in particular a current stress value, which is represented graphically for the medical service personnel in a further step 103 via the display unit 22 of the magnetic resonance device. In this connection the display unit 22 can be arranged in a control room and/or a monitoring room which is constructed separate from an examination room.

In a further step 104 the calculated emotional state, in particular the stress value of the patient 15 is compared to a first threshold value. The first threshold value can be read from a database, wherein in this connection the step 104 is performed by the system control device 21. As an alternative, it is also possible that the first threshold value is made up of empirical values of the medical service personnel and the step 104 is carried out manually by the medical service personnel.

If the calculated emotional state, in particular the stress value, of the patient 15 lies below this first threshold value, no changes and/or adjustments will be made in a measurement protocol during the medical imaging examination. Also, no sedation steps will be carried out to calm the patient 15. Also, the surroundings of the patient 15 remain unchanged. If the medical imaging examination is not yet finished, which is determined in a query 105, emotion parameters are again recorded by means of the sensor unit 24 in step 101 and the subsequent steps are carried out again.

If the calculated emotional state, in particular the stress value of the patient 15 lies above this first threshold value, in a further step 106 the calculated emotional state, in particular the stress value of the patient 15 is compared with a second threshold value. The second threshold value can be read from a database, wherein in this connection the step 106 is carried out by the system control device 21. As an alternative it is also possible that the second threshold value is made up of empirical values of the medical service personnel and the step 106 is performed manually by the medical service personnel.

Information to the effect that the calculated emotional state, in particular the stress value of the patient 15 lies above the first threshold value can be graphically represented in step 106 also via the display unit 22 and in this way be communicated to the medical service personnel.

If the calculated emotional state, in particular the stress value of the patient 15 lies below this second threshold value, at least a first sedation step 107 is carried out, wherein in the first sedation step 107 an adjustment and/or a change of the surroundings of the patient 15 takes place. In this first sedation step 107 reassuring words can be spoken to the patient 15 by the medical service personnel via the communication unit not presented in detail. In addition, in the first sedation step 107 a lighting and/or illumination of the patient reception area can be adjusted and/or changed by means of the lighting unit 14, so that the lighting and/or illumination of the patient reception area 14 has a calming effect on the patient 15. In addition, in the first sedation step 107 provision can also be made that music having a calming effect on the patient 15 is played in the patient reception area 14 and/or via headphones. Further, the ventilation can be adapted to the stress value of the patient 15 by means of the patient reception area 14, wherein the ventilation of the patient reception area 14 is carried out by means of a ventilation unit of the magnetic resonance device not presented in detail. In addition, further measures for calming the patient 15 that seem practical to a person skilled in the art are conceivable at any time.

This first sedation step 107 can be carried out manually by the medical service personnel. In this connection, provision can additionally be made that, for support of the medical personnel an instruction and/or a selection of instructions is graphically presented on the display unit 22 in the first sedation step 107, wherein the instruction appears on the display unit 22 controlled by the system control device 21. As an alternative it is also possible for the first sedation step 107, or the calming measures initiated to calm the patient 15 to be carried out at least partially automatically by the system control device 21 or are controlled by the system control device 21.

If the medical imaging examination is not yet finished, which is determined in the query 105, after execution of the first sedation steps 107 emotion parameters are again recorded by means of the sensor unit 24 in step 101 and the following steps are carried out again.

If the calculated emotional state, in particular the stress value of the patient 15 lies above this second threshold value, at least a second sedation step 108 is carried out. In this second sedation step 108 additional calming measures take place to calm the patient 15. Thus, in the second sedation step 108 measurement parameters of the medical imaging examination are adjusted to the calculated emotional state, in particular the stress value of the patient 15 and/or changed.

A change and/or adjustment of measurement parameters of the medical imaging examination in the second sedation step 108 means a change and/or an adjustment of measurement parameters within a sequence of a measurement protocols and/or scan protocol of the medical imaging examination. This change and/or adjustment of measurement parameters within the measurement protocol in the second sedation step 108 can in this connection for example comprise a change and an adjustment with respect to the magnetic resonance sequences that have been selected or that are available for selection. Thus, for example only those magnetic resonance sequences can be selected and/or available for selection for the purpose of reassuring the patient that facilitate an activation of a whisper mode, so that on the basis of the low-noise magnetic resonance sequences, a calming effect is exerted on the patient 15 in the further course of the medical imaging examination. In addition, this change and/or adjustment of measurement parameters within the measurement protocol in the second sedation step 108 can include a selection of magnetic resonance sequences, designed to be desensitized vis-à-vis a movement of the patient 15 and/or to make possible a faster measurement sequence, so that a stressful situation for the patient 15 can be shortened.

Further this change and/or adjustment of measurement parameters within the measurement protocol in the second sedation step 108 can also lead to a pause in the measurement. In addition, an adjustment and/or change of a Dot (Day optimizing throughput) engine strategy is also possible, wherein a Dot engine strategy optimizes a workflow for magnetic resonance devices in particular.

Furthermore, provision can be made that in the second sedation step 108 the change and/or adjustment of measurement parameters within the measurement protocol comprises cancelling at least a partial measurement of the medical imaging examination, as is of advantage in particular in the case of very high stress values of the patient 15. In addition, provision can be made that in the second sedation step 108 the patient 15 is removed from the patient reception area 14, as is of advantage in particular in the event of panic attacks and/or in the event of looming panic attacks of the patient 15.

This second sedation step 108 can be carried out manually by the medical service personnel. In this connection, provision can additionally be made that for support of the medical service personnel an instruction and/or a selection of instructions are graphically presented on the display unit 22 in the second sedation step 108, wherein the instruction is displayed on the display unit 22 controlled by the system control device 21. As an alternative, it is also possible for the second sedation step 108, or the calming measures initiated for reassuring the patient, to be carried out at least partially automatically by the system control device or are controlled by the system control device 21.

Information to the effect that the calculated emotional state, in particular the stress value, of the patient 15 lies below the second threshold value or above the second threshold value can also be graphically displayed in the step via the display unit 22 and in this way be communicated to the medical service personnel.

In addition, it is also conceivable for an adjustment and/or a change of measurement parameters to take place already in the first sedation step 107 within a sequence plan of the measurement protocol of the medical imaging examination.

In addition, provision can also be made that in the second sedation step 108 measures are taken and or executed for changing and/or adjusting the environment of the patient 15.

Although modifications and changes may be suggested by those skilled in the art, it is the intention of the inventors to embody within the patent warranted hereon all changes and modifications as reasonably and properly come within the scope of their contribution to the art.

We claim as our invention:

1. A method for representing an emotional state of a patient during a medical imaging examination, comprising:
with a physiological detector that interacts with a patient undergoing a medical imaging examination, detecting at least one physical characteristic of the patient during the medical imaging examination, which is indicative of emotional stress being experienced by the patient during the medical imaging examination;
supplying the detected parameter to a processor during said medical examination and, in said processor, automatically calculating a stress value that represents a quantification of said emotional stress being experienced by the patient; and
making the calculated stress value available from the processor in a humanly-perceptible form during the medical imaging examination.

2. A method as claimed in claim 1 comprising detecting said physiological parameter as skin conductivity of the patient with said detector placed on the skin surface of the patient.

3. A method as claimed in claim 1 comprising representing said calculated stress value in humanly-perceptible form by representing said calculated stress value graphically at a display in communication with said processor.

4. A method as claimed in claim 1 comprising sedating the patient dependent on the value of said calculated stress value.

5. A method as claimed in claim 4 comprising, when sedating a patient, adjusting or changing measurement parameters used to implement said medical imaging examination.

6. A method as claimed in claim 5 comprising, while sedating the patient, canceling at least a portion of said medical imaging examination.

7. A method as claimed in claim 4 comprising sedating the patient by adjusting or changing an environment of the patient dependent on the calculated stress value.

8. A method as claimed in claim 4 comprising manually executing sedation of the patient.

9. A method as claimed in claim 4 comprising graphically presenting, at a display in communication with said processor, an instruction for executing sedation of the patient.

10. A method as claimed in claim 4 comprising at least partially automatically implementing sedation of the patient.

11. A medical imaging device comprising:
a medical imaging data acquisition unit adapted to acquire image data from a patient in a medical imaging examination;
a physiological detector that interacts with the patient undergoing the medical imaging examination, to detect at least one physical characteristic of the patient during the medical imaging examination, which is indicative of emotional stress being experienced by the patient during the medical imaging examination;
a processor supplied with the detected parameter during the medical examination, said processor being configured to automatically calculate a stress value that represents a quantification of said emotional stress being experienced by the patient; and
said processor being configured to make the calculated stress value available from the processor in a humanly-perceptible form during the medical imaging examination.

12. A medical imaging device as claimed in claim 11 comprising an emergency call device configured to be activated by the patient during the medical imaging examination, said physiological detector being integrated into said emergency call device.

13. A medical imaging device as claimed in claim 11 wherein said physiological detector is a conductivity sensor adapted for placement on the skin surface of the patient.
